(19) United States
(12) Patent Application Publication
(10) Pub. No.: US 2016/0161425 A1
(43) Pub. Date: Jun. 9, 2016

(54) APPARATUS AND METHOD FOR DETERMINING A VALUE OF A PROPERTY OF A MATERIAL USING MICROWAVE

(71) Applicant: GOJI LIMITED, Hamilton (BM)

(72) Inventors: Maksim BEREZIN, Netanya (IL); Ram ELBOIM, Modiin (IL); Eli DICHTERMAN, Haifa (IL); Ben ZICKEL, Qiryat Bialik (IL)

(73) Assignee: GOJI LIMITED, Hamilton (BM)

(21) Appl. No.: 14/787,606

(22) PCT Filed: May 2, 2014
(86) PCT No.: PCT/EP2014/059014

§ 371 Date: Oct. 28, 2015

Related U.S. Application Data
(60) Provisional application No. 61/845,415, filed on Jul. 12, 2013.
(60) Provisional application No. 61/819,042, filed on May 3, 2013.

(51) Int. Cl. G01N 22/04; G01N 33/28; G01F 1/66

(52) U.S. Cl. CPC G01N 22/04; G01F 1/66; G01N 33/2823; G01N 33/2841; G01N 33/2847

(57) ABSTRACT

Methods and apparatuses for determining a value of a property of a material that flows in a conduit inside a microwave cavity are described. Such apparatus may include: a multi-mode microwave cavity having the conduit in it; a plurality of feeds, each configured to feed the cavity with RF radiation to excite multiple modes in the cavity; a detector, configured to detect parameters indicative of electrical response of the cavity to RF radiation fed to the cavity; and a processor, configured to determine the value of the property based on the parameters detected by the detector. In some embodiments, at least one of the feeds comprises a radiating element outside the cavity and a waveguide configured to guide waves from the radiating element to the cavity.

### CROSS-REFERENCE TO RELATED APPLICATIONS

The present application claims the benefit of each of U.S. Provisional Pat. Appl. No. 61/819,042, filed on May 3, 2013, and U.S. Provisional Pat. Appl. No. 61/845,415, filed on Jul. 12, 2013. The disclosures of each of which, including the specifications, claims, and figures, are incorporated herein by reference in their entireties.

### FIELD AND BACKGROUND

The present application is in the field of investigating materials by the use of microwave. More particularly but not exclusively, some embodiments are in the field of investigating phase composition of crude oil or other multi-phase materials.

Proposals to investigate multi-phase materials using microwave have been made at least since the 1970s, but to the best of the knowledge of the inventors, such proposals never matured into a commercially available product. Accordingly, the inventors believe that the field may benefit from a new approach.

### SUMMARY

According to some embodiments of the invention, there is provided an apparatus for determining a value of a property of a material that flows in a conduit inside a microwave cavity. The apparatus may include:

a multi-mode microwave cavity having therein the conduit;

a plurality of feeds, each configured to feed the cavity with RF radiation to excite multiple modes in the cavity;

a detector, configured to detect parameters indicative of electrical response of the cavity to RF radiation fed to the cavity; and

a processor, configured to determine the value of the property based on the parameters detected by the detector. In some embodiments, at least one of the feeds comprises a radiating element outside the cavity and a waveguide configured to guide waves from the radiating element to the cavity.

In some embodiments, the apparatus may further comprise an attenuator attached to the cavity. The attenuator may be configured to attenuate electrical field exiting from the cavity, for example, to less than 1% of the electrical field inside the cavity.

In some embodiments, the attenuator may comprise an RF reflective attenuating conduit portion. In some embodiments, a dielectric attenuating conduit portion may be attached to the RF reflective attenuating conduit portion.

In some embodiments, the attenuating conduit may be arranged such that the field intensity at the end of each dielectric conduit portion far from the metallic conduit portion is smaller than 1% of the field intensity inside the cavity.

In some embodiments, the attenuator may be arranged not to interfere with flow of the material.

In some embodiments, the parameters indicative of electrical response of the cavity to RF radiation fed to the cavity may include a ratio of power measured to get back from the cavity at a given feed to power measured to go towards the cavity at the given feed. For example, the parameters indicative of electrical response of the cavity to RF radiation fed to the cavity may include a scattering parameter S₁₁.

In some embodiments, the feeds are isolated from each other. For example, the isolation between the fields may be such that in exciting most of the modes (e.g., 60%, 70%, or 80%, of the modes) in the cavity, less than 10% of power entering the cavity through one feed exits the cavity through another feed. In some embodiments, the isolation is such that in response to most of the frequencies, less than 10% of power entering the cavity through one feed exits the cavity through another feed. In some embodiments, the isolation is such that in exciting most modes (or, in some embodiments, upon irradiating with most of the frequencies), less than 10% of the power entering through one feed exits the cavity through all the other feeds together.

In some embodiments, less than 10% of the power entering through one feed exits the cavity through the other feeds when the cavity is full with a material having the same dielectric constant as the conduit.

In some embodiments, one of the feeds is inclined in respect of a symmetry axis of the cavity. In some embodiments, two or more of the feeds are so inclined. In some embodiments two feeds may be spaced apart from one another such that electromagnetic radiation propagating along an axis of symmetry of one feed and reflected from an inner face of the cavity propagates out of the cavity through another feed. In some embodiments, the two feeds may be equally inclined.

In some embodiments, the feeds may comprise a pair of inclined parallel feeds. In some embodiments, two or more pairs of inclined parallel feeds may be included in the apparatus. The symmetry axes of the inclined parallel feeds may be non-overlapping with each other.

In some embodiments, a feed may include a radiating element having an end, and a waveguide for guiding waves from the end of the radiating element to the cavity. In some such embodiments, the end of the radiating element may be distanced from the cavity by half a wavelength or more. The wavelength may be the wavelength in the waveguide of the lowest frequency of the microwave radiation exciting the modes in the cavity.

In some embodiments, the material under investigation may be familiar in the sense that the material has a dielectric constant within a given range. In some such embodiments, the conduit (within which the material may flow in operation) may be made of a dielectric material having a dielectric constant within the said given range. For example, the conduit may be made of a material having a dielectric constant within a lower half of the said given range.

In some embodiments, the waveguide may have a cutoff frequency that is lower than or equal to the cutoff frequency of the cavity.

In some embodiments, a diameter of the waveguide is half or less a diameter of the cavity.

In some embodiments, the processor may be configured to determine the value of the property of the material under investigation by applying a kernel method to measurement results associating frequencies (or other excitation setups) with values of parameters indicative of the electrical response of the cavity to the excitation.

In some embodiments, the parameters indicative of electrical response of the cavity to the excitation of the modes in the cavity include a ratio of power measured to get back from the cavity at a given feed to power measured to go towards the cavity at the given feed. For example, the parameters indicative of electrical response of the cavity to the excitation of the modes in the cavity include a scattering parameter S₁₁.

In some embodiments, the values of parameters indicative of the electrical response of the cavity to the excitation may include values measured by the detector.

In some embodiments, the processor may be configured to combine parameters measured by the detector to obtain combined parameters. The processor may be further configured to determine the property based on the combined parameters. In some embodiments, each combined parameter is associated with one of the feeds. Examples of parameters that may be used by the processor for determining the value of the property of the object may include s parameters, Γ parameter, and dissipation ratios.

According to some embodiments of the invention, there is provided a method of determining a value of a property of a material that flows in a conduit inside a microwave cavity. The method may include:

exciting multiple modes in the cavity through a number of feeds;

detecting parameters indicative of electrical response of the cavity to the excitation of the modes in the cavity; and

determining the value of the property based on the detected parameters.

In some embodiments, the method may include:

Irradiating microwave radiation into the cavity at a plurality of excitation setups, each defining a set of controllable parameters that affect a field pattern excited in the cavity;

Detecting parameters indicative of electrical response of the cavity to the irradiated microwaves; and

Determining the value of the property based on the detected parameters.

In some embodiments, at least one of the feeds comprises a radiating element outside the cavity and a waveguide configured to guide waves from the radiating element to the cavity.

In some embodiments, the excitation of the multiple modes in the cavity may include excitation of a number of modes that is larger than the number of feeds.

In some embodiments, the determination of the value of the property may include application of kernel methods. These methods may be applied to parameters measured by the detectors. In some embodiments, the kernel methods may be applied to combined parameters. The combined parameters may be combinations of the measured parameters. In some embodiments, these combinations may be linear. In some embodiments, these combinations may be non-linear.

Accordingly, in some embodiments, determining the value of the property comprises combining parameters measured by the detector to obtain combined parameters, and determine the property based on the combined parameters.

In some embodiments, the method may include operating an apparatus, which by itself is according to some embodiments of the present invention.

In some embodiments, exciting a number of modes is by applying to the cavity RF radiation at a plurality of excitation setups. In some embodiments, each two of the excitation setups differ from one another in at least one of a frequency or a feed, through which RF radiation is fed to the cavity to obtain the excitation.

In some embodiments, the material is a multi-phase material, for example, crude oil or milk. In some such embodiments, the property may be a phase-composition of the material, or a volume fraction of one of the material's components, for example, the volume fraction of water in the material, the volume fraction of oil in the material, etc.

According to an aspect of the present disclosure, an apparatus is provided for determining a flow rate of a multi-phase material that flows in a conduit of a microwave cavity. The apparatus may include a multi-mode microwave cavity through which the conduit extends, a plurality of inclined parallel feeds, each of the feeds configured to deliver RF radiation to the cavity to excite multiple modes in the cavity, each of the feeds comprising a radiating element exterior to the cavity and a waveguide configured to guide electromagnetic waves from the radiating element to the cavity, a detector that detects parameters indicative of an electrical response of the cavity to RF radiation delivered to the cavity, and a processor, that determines the flow rate of the multi-phase material based upon the parameters detected by the detector. The apparatus may also include an attenuator having an RF reflective attenuating conduit portion and a dielectric attenuating conduit portion. The apparatus may also include a plurality of attenuators, in which each of the plurality of attenuators has an RF reflective attenuating conduit portion and a dielectric attenuating conduit portion. In another aspect, an attenuator may have a metallic attenuating conduit portion and a dielectric attenuating conduit portion. Further, a plurality of attenuators may be provided in which each of the plurality of attenuators has a metallic attenuating conduit portion and a dielectric attenuating conduit portion.

Still further, the apparatus may include a pressure sensor configured to measure differential pressure of the multi-phase material and a temperature sensor configured to measure a temperature of the multi-phase material. Yet further, the apparatus may include an inlet to the cavity and an outlet to the cavity, in which at least one of the inlet and the outlet is at least partially covered by a net. In one aspect, the net contains a metallic material.

Further, different frequencies can be applied to each of the plurality of inclined feeds at the same time. Additionally, excitation can be applied to less than all of the plurality of inclined parallel feeds at a given time. In one aspect the multi-phase material includes a wet gas and/or crude oil.

According to another aspect of the present disclosure, a method is provided for determining a flow rate of a multi-phase material that flows in a conduit of a microwave cavity. The method includes exciting multiple modes in the microwave cavity through a plurality of inclined parallel feeds, in which each of the feeds includes a radiating element exterior to the cavity and a waveguide configured to guide electromagnetic waves from the radiating element to the cavity. The method also includes detecting parameters indicative of an electrical response of the cavity to RF radiation delivered to the cavity. Additionally, the method includes determining the flow rate of the multi-phase material based upon the parameters detected by the detector. The method may include detecting an object flowing in the multi-phase material. The method may also include measuring reflected signals from the multi-phase material to identify a substance foreign to the multi-phase material. The method may also include analyzing the reflected signals to identify a substance foreign to the multi-phase material. Still further, the method may include detecting a flow rate of a gas flowing in the multi-phase material. Yet further, the method may include measuring a differential pressure of the multi-phase material and measuring a temperature of the multi-phase material.

Further, the method may include transmitting an alarm signal upon a detection of a foreign substance in the multi-phase material. In one aspect the multi-phase material includes a wet gas and/or crude oil. Further, the method may include applying different frequencies to each of the plurality of inclined parallel feeds at the same time. Still further, the method may include applying excitation to less than all of the plurality of inclined parallel feeds at a given time.

An aspect of some embodiments of the invention relates to a method of determining a value of a property of a material that flows in a conduit inside a microwave cavity. The method may include:
exciting a plurality of excitation setups in the microwave cavity through a plurality of feeds;
detecting parameters indicative of electrical response of the microwave cavity to the excitation of the excitation setups in the microwave cavity; and
determining the value of the property based on the detected parameters by application of a kernel method.

In some embodiments, each two of the plurality of excitation setups differ from one another in at least one of a frequency or a feed.

In some embodiments, the parameters indicative of electrical response of the microwave cavity to the excitation of the excitation setups in the microwave cavity include a scattering parameter S₁₁.

In some embodiments, material is crude oil.

In some embodiments, the property includes a volume fraction of water in the material, a volume fraction of oil in the material and/or a volume fraction of gas in the material.

An aspect of some embodiments of the invention may relate to and apparatus for determining a value of a property of a material that flows in a conduit inside a microwave cavity. The apparatus may include:
a multi-mode microwave cavity having therein the conduit;
a plurality of feeds, each configured to feed the microwave cavity with RF radiation to excite multiple excitation setups in the microwave cavity;
a detector, configured to detect parameters indicative of electrical response of the microwave cavity to radio frequency (RF) radiation fed to the microwave cavity; and
a processor, configured to determine the value of the property based on the parameters detected by the detector by application of a kernel method.

An apparatus as described above may be adapted to carry out any one of the above-mentioned methods.

Unless otherwise defined, all technical and/or scientific terms used herein have the same meaning as commonly understood by one of ordinary skill in the art to which the invention pertains. Some methods and materials that can be used in the practice or testing of embodiments of the invention are described below. Yet, other or equivalent materials and methods can be used in the practice or testing of embodiments of the invention. In addition, the materials, methods, and examples are illustrative only and are not intended to be necessarily limiting.

Implementation of the method of embodiments of the invention can involve performing or completing selected tasks automatically. Moreover, according to actual instrumentation and equipment of embodiments of methods of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments described herein are performed by a data processor, such as a computing platform for executing a plurality of instructions. In some embodiments, the data processor includes a volatile memory for storing instructions and/or data. In some embodiments, the data processor may include a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A is a diagrammatic representation of an apparatus according to some embodiments of the invention;

FIGS. 1B and 1C are isometric views of two cavities with feeds according to some embodiments of the invention;

FIG. 2 is a diagrammatic illustration of a cavity with isolated feeds according to some embodiments of the invention;

FIG. 3 is a flow chart of a method of determining a value of a property of a material according to some embodiments of the invention;

FIG. 4A is a diagrammatic presentation of an apparatus with an attenuator according to some embodiments of the invention,

FIG. 4B is a diagrammatic presentation of a front view of the attenuator shown in FIG. 4A;

FIG. 5A is a diagrammatic presentation of an apparatus with an attenuator according to some embodiments of the invention;

FIG. 5B is a diagrammatic presentation of an apparatus with an attenuator according to some embodiments of the invention;

FIG. 5C is a diagrammatic illustration of a front view of the attenuator shown in FIG. 5B;

FIG. 6 is a diagrammatic illustration of an apparatus with an attenuator according to some embodiments of the invention;

FIG. 7 is a diagrammatic illustration of a multi-phase flow meter, according to some embodiments of the present disclosure;

FIG. 8 is an exemplary architecture within which the multi-phase flow meter is used, according to some embodiments of the present disclosure; and

FIG. 9 shows an exemplary general computer system that includes a set of instructions for the multi-phase flow meter, according to an aspect of the present disclosure.

### DETAILED DESCRIPTION

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments and may be practiced or carried out in various ways.

The present disclosure is in the field of investigating materials by the use of microwave. More particularly but not exclusively, some embodiments are in the field of investigating phase composition of crude oil or other multi-phase materials.

An aspect of some embodiments of the invention includes an apparatus for determining a value of a property of a material. For example, the property may be a volume fraction of water, and the value may be 5%. The material may include a plurality of phases, for example, the material may be crude oil, comprising oil and water; milk, comprising water and fat, or any other multi-phase material. Preferably, at least one of the phases has dielectric properties distinctive from the other phases.

The property to be investigated may be any property that affects the dielectric constant of the material, for example, volume fraction of any one of the phases, temperature of the material, chemical composition of the material (e.g., salts dissolved in one of the phases), presence of metals or other foreign bodies, etc. For example, in some embodiments it would be desirable to detect the presence and percentages of substances in petroleum products, including crude oil, as such substances can be harmful, cause pollution, and create inefficient burning. Accordingly, the properties to be investigated may include polyaromatic hydrocarbons (PAH), sulfur containing organic materials content, hydrogen sulfide content, nitrogen containing organic materials, tars, and other carbonaceous materials.

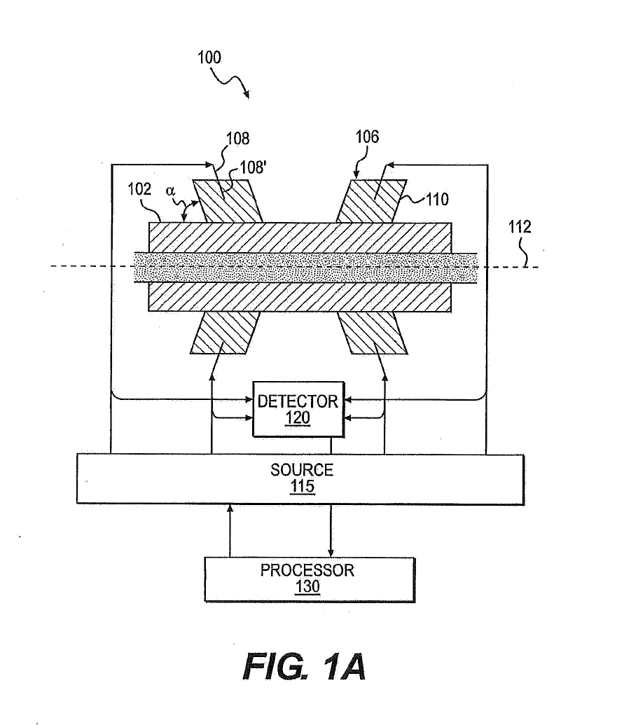

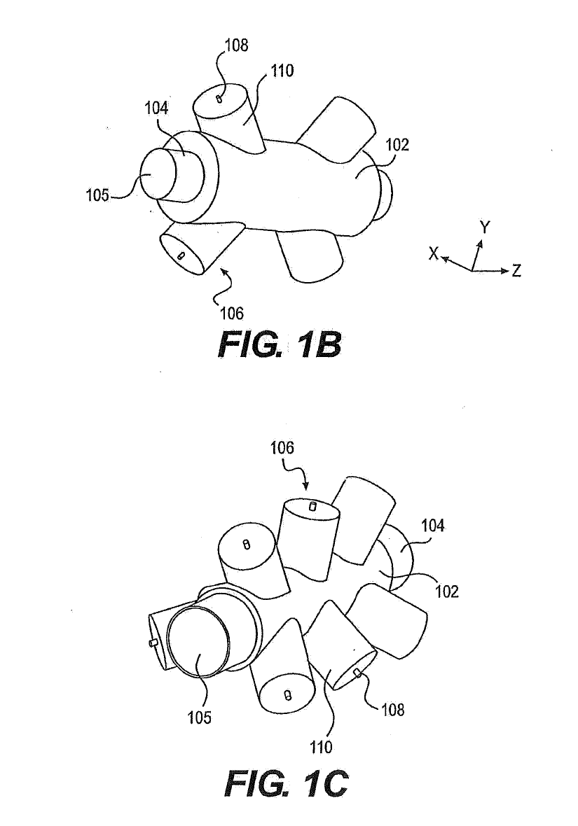

In operation, the material may flow in a conduit inside a microwave cavity. FIG. 1A is a diagrammatic representation of an apparatus 100 according to some embodiments of the invention. Illustrated in FIG. 1A are the cavity (102), the conduit (104), inside which the material under investigation (105) may flow in operation, a plurality of feeds 106 for feeding the cavity with RF energy, a detector 120 for detecting the electrical response of the cavity with the material flowing therein to microwave radiation fed through the feeds, and a processor 130, for determining the property of the material based on the readings of the detector.

In the following description conduit 104 is considered to be cylindrical and to have its axis of symmetry overlap with a symmetry axis (112) of a cylindrical cavity 102, but other constructions are also possible. For example, the cavity may have any shape, for example, it may be cylindrical, prismatic, rectangular, etc. In some embodiments, the cavity may have the same symmetry as the conduit, for example, a cylindrical cavity may be used with a cylindrical conduit, a rectangular cavity with a rectangular conduit, etc. In some embodiments, the symmetry of the cavity may differ from that of the conduit. In some embodiments, the conduit may be positioned along a longitudinal axis (e.g., symmetry axis) of the cavity. In some embodiments, the conduit may run in parallel to a longitudinal axis of the cavity, or it may be non-parallel to the said axis. In some embodiments, the conduit 104 has a diameter between one inch and eight inches; although, up to at least twenty-four inches is also contemplated. Smaller diameters, for example of 100 microns may also be contemplated. In some embodiments, the conduit may be adapted to handle flow rates of about 1 m/sec, so a one to two inch diameter conduit may fit to about 50-500 barrels of oil per day, or an equivalent amount of gas such as natural gas. Further, in some embodiments, the conduit 104 is capable of withstanding pressures of at least 50 bar, in some embodiments at least 250 bar. In some embodiments, the conduit 104 is capable of withstanding temperatures of at least 150° C., in some embodiments, at least 250° C.

In some embodiments, conduit 104 may be made of a material having a dielectric constant ∈_{conduit} that is the same as the dielectric constant of the material under investigation ∈ₘₐₜₑᵣᵢₐₗ. Since ∈ₘₐₜₑᵣᵢₐₗ may depend on the property of the material, it is generally unknown. However, it may be known that ∈ₘₐₜₑᵣᵢₐₗ is expected to lie within a certain range. In some embodiments, ∈_{conduit} has a value inside that certain range. In some embodiments, it may be preferred to have a conduit with ∈_{conduit} in the lower half of the range, for example, if the range of values that the dielectric constant of the material may have is between 1.5 and 5, the conduit may be made of a material having a dielectric constant between 1.5 and 3.25, for example, 2, 2.2, 2.5, 3, etc. In a particular example, a conduit for crude oil (2<∈material<5) may be made of Teflon (∈conduit=2.2).

In some embodiments, the cavity may be open-ended, so material may flow freely in and out of the cavity without requiring opening and closing doors or valves. The cavity may support standing waves in the frequency range used for investigating the material. It is noted, however, that the open ends may allow some of the radiation applied to the cavity for investigating the material to escape from the cavity.

The cavity may be multi-mode in the sense that it may support multiple modes in the range of frequencies used for the determination of the material's property. The range of frequencies that may be used to investigate the material may be all above a cutoff frequency of the cavity, and may be as broad as possible, since it is suggested that in some embodiments of the present invention accuracy may be improved by enlarging the number of modes excited in the cavity during investigation. In some embodiments, the frequency range may be between 1.5 GHz and 5.5. GHz, between 2 GHz and 8 GHz, between 500 MHz and 1000 MHz, or any other portion of the microwave frequency range, that is between 300 MHz and 300 GHz. In some embodiments, lower frequencies (e.g., 10 MHz to 300 MHz) may be used, and the term “microwave” as used herein may include them too. In some embodiments, the frequency range may include at least two octaves (i.e., the highest frequency is at least four times higher than the lowest frequency). For example, in such embodiments, if the lowest frequency is 1 GHz, the highest frequency is at least 4 GHz. In some embodiments, the frequency range may have a width (i.e., breadth) of at least 100% of the central frequency (i.e., the difference between the highest and lowest frequency is at least as large as the central frequency). For example, in such embodiments, if the central frequency is 2 GHz, the frequency range may be between 1 GHz and 3 GHz, or any other broader frequency range centered around 2 GHz.

Apparatus 100 may have a plurality of feeds 106. In some embodiments, accuracy of the investigation may be higher with apparatuses having a larger number of feeds. For example, an apparatus with four feeds (As shown in FIG. 1A) may provide higher accuracy than a similar apparatus with 3 feeds, two feeds, or a single feed, and an apparatus with a larger number of feeds, e.g., 9 feeds, may allow higher accuracy than a four-feed apparatus. The number of feeds may affect the number of modes that may be excited in the cavity, and may also affect the spatial distribution of local intensity maximums of the excited modes. The local intensity maximums may be important, since the readings of the detector may be more strongly affected by properties of the material in the vicinity of such maximums than away of such maximums.

Excitation of each mode generates in the cavity a typical electrical field distribution (also referred to herein as a field pattern). The field pattern may have one or more local extremum points, at which the field amplitude is at minimum or maximum, and the field intensity is at maximum. Having more feeds may allow exciting in the cavity modes having their local intensity maximums more widely distributed inside the cavity. For example, each mode is most easily excitable by a feed that lies at an intensity maximum of the field pattern associated with the mode. Accordingly, having feeds in many different places may facilitate exciting in the cavity modes having their intensity maximums at many different places. It is suggested herein that wide spread of local intensity maximums within the investigated material may enhance the accuracy.

In some embodiments, accuracy may be optimized by exciting in the cavity such modes, that their local intensity maximums cover the entire volume of the material under investigation. For example, each local intensity maximum may be associated with a volume around the maximum, at which the field intensity is larger than half the intensity at the maximum. In some embodiments, the volumes associated with all the local intensity maximums of all the modes excited in the cavity cover the entire volume of the material under investigation flowing inside the cavity. The volume of the material under investigation 105 is the volume in the void defined by the walls of conduit 104 inside cavity 102. In some embodiments, optimal locations may be determined for feeds of a given number by calculating, e.g., from a simulation, for each set of locations, the total volume of the local intensity maximums of the field patterns excitable in the cavity by the feeds at the tested locations. A location set at which this volume is maximal among the tested sets may be used in practice to maximize the coverage of the material under investigation with local maximums.

In some embodiments, one or more of the feeds 106 comprises a radiating element 108, outside cavity 102 and a waveguide 110 configured to guide waves from radiating element 108 to the cavity. Having radiating elements 108 outside cavity 102 may reduce direct coupling between the feeds 106. In some embodiments, radiating element 108 may have an end 108′, through which microwave radiation may emanate. In some embodiments, the wall of cavity 102 may have an opening 102′ for receiving radiation from feed 106. Opening 102′ may fit the outer shape of waveguide 110. In some embodiments, the distance between end 108′ and opening 102′ may be λ/2, wherein λ is the wavelength, inside waveguide 110, of the lowest frequency used for investigating the material (i.e., the lowest frequency of the RF radiation exciting modes in the cavity). Waveguide 110 may be filled with a dielectric material having a dielectric constant ∈waveguide. In some embodiments, the filling of waveguide 110 may be chosen to ensure that the cutoff frequency of waveguide 110 is not higher than the cutoff frequency of cavity 102. In some embodiments, the physical dimensions of waveguide 110, e.g., its diameter, and the dielectric constant ∈waveguide are such that the diameter of the waveguide is about half that of the cavity, or less, for example, the ratio between the diameters may be between 0.25 and 0.5. Some values of dielectric constants for the filler of the waveguide may be, for example, 6, 9, or 12.

In some embodiments, feeds 106 may be isolated from each other. It was found by the inventors that better isolation may bring about higher accuracy. The inter-feed isolation may vary across frequencies, and in some embodiments, frequencies at which the isolation is below a threshold may be discarded, for example, they may be disregarded by processor 130 when the property is determined. Minimizing inter-feed coupling may be another way to improve accuracy of the apparatus. Thus, in some embodiments, the isolation between the feeds is such that less than 10% of power entering the cavity through one feed exits the cavity through another feed. In some embodiments, the isolation between the feeds is such that less than 10% of power entering the cavity through one feed exits the cavity through all the other feeds together. In some embodiments, these levels of isolation may be kept only across some of the frequencies, for example, across half or more, 75% or more or 80% or more of the frequencies used for determining the value of the property. In some embodiments, ‘frequencies used’ may include only frequencies used by processor 130 for determining the property. In some embodiments, ‘frequency used’ may include all the frequencies at which radiation is fed into cavity 102 for the investigation.

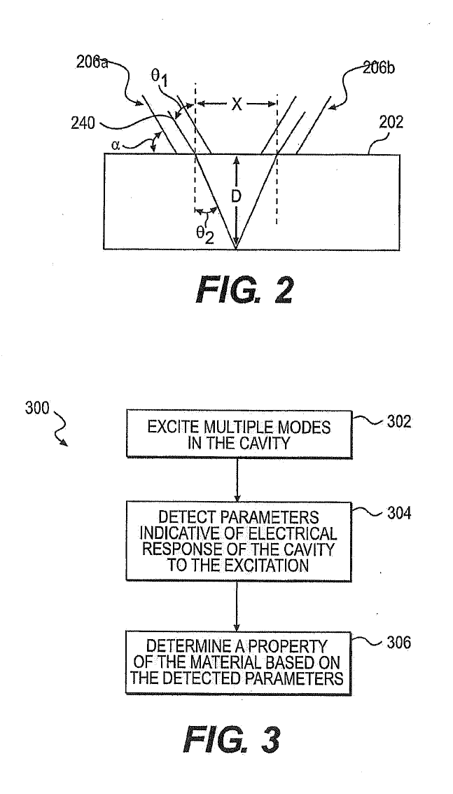

In some embodiments, inter-feed isolation may be enhanced by properly spacing and/or orienting the feeds. For example, in some embodiments, at least one of the feeds is inclined in respect of a symmetry axis of the cavity. This may be exemplified in FIG. 1A by feeds 106 being inclined in respect of symmetry axis 112. One way of optimizing inter-feed isolation according to some embodiments is discussed below with reference to FIG. 2 in the context of inclined feeds. The inclination angle α may be, for example, between 20° and 70°, for example, 30°, 40°, 45°, 50°, 60°, or any other intermediate angle. One or more of the feeds may be perpendicular to the axis (e.g., α may be 90°, optionally 90°±10°). Inclined feeds may be advantageous over perpendicular feeds in that they may allow exciting, by a single feed, modes of different types, for example, TE, TM, and quasi-TEM. In some embodiments, the feeds may include one or more pairs of parallel feeds. Parallel feeds may be feeds, each having a symmetry axis, wherein the symmetry axes of the feeds are substantially parallel to each other. For example, the angle between them may be smaller than 10°, preferably around 0°. In some embodiments, feeds with parallel symmetry axes may be positioned such that their symmetry axes overlap. However, to improve decoupling between the feeds it may be preferable to have the parallel feeds with non-overlapping symmetry axes, e.g., inclined parallel feeds that do not overlap. Such two pairs of parallel feeds with non-overlapping symmetry axes are illustrated in FIG. 1A, where feeds that lie diagonally to each other are parallel to each other. In some exemplary embodiments, two of the feeds are equally inclined with respect to the axis of symmetry of the cavity, (e.g., one extends at 40° to the symmetry axis, and the other extends at 140° to the symmetry axis) and are spaced apart from one another such that electromagnetic radiation propagating along an axis of symmetry of one feed and reflected from an inner face of the cavity propagates out of the cavity through the other of the two feeds. The equally inclined feeds may lie on a line parallel to the symmetry axis of the cavity. In some embodiments, the equally inclined feeds may lie off set from one another, for example, on a line non parallel to the symmetry axis of the cavity.

FIG. 1B is an isometric view of a cavity according to some embodiments of the invention. FIG. 1A shows a cavity 102 with four feeds 106. The feeds shown in FIG. 1B are all on the same plane. Each feed 106 is shown to include a radiating element 108 and waveguide 110. Radiating element 108 may penetrate into waveguide 110, but this is not seen in the present view. Also shown in the figured are the material to be investigated (105) and a dielectric conduit 104, within which material 105 may flow. In some embodiments, the dielectric conduit fills the entire cavity, other than space left for the material to be investigated, as shown diagrammatically in FIG. 1A.

FIG. 1C is an isometric view of a cavity according to some embodiments of the invention. In FIG. 1C a cavity (102) with nine feeds (106) is shown. The feeds are arranged in groups of three. The group in the middle comprises feeds that are on a plane perpendicular to the symmetry axis of conduit 104. The groups at the edge, each comprises three pairs of feeds, and each pair is on a plane inclined to the symmetry axis of conduit 104 and non-parallel to any of the other two planes. Orienting the feeds on such non-parallel planes may increase inter-feed isolation, and thus, in some embodiments, may enhance accuracy.

Some embodiments, such as those depicted in FIGS. 1A-1C may include a pair of inclined parallel feeds. The parallel feeds may be coplanar, for example, the central symmetry axis of the feeds may lie on the same plane. In some embodiments, the central symmetry axis of the feeds may be parallel or substantially parallel (e.g., be inclined one in respect of the other by 10° or less, 5° or less, or 2° or less. In some embodiments, the parallel axes do not overlap, so that despite of the feeds being parallel, a ray going in straight line along the symmetry axis of one of the feeds will not enter the other feed.

In some embodiments, the inclination angle α (see FIG. 1A) and the distance X between two inclined feeds may be such that there is high coupling between the feeds at one particular mode, and low coupling at all other modes. One way to achieve this effect is illustrated in FIG. 2. FIG. 2 is a diagrammatic illustration of a cavity 202 with two feeds 206a and 206b. For simplicity, a conduit for the material is not shown. Similarly, additional feeds are not shown for the sake of simplicity. The diameter of cavity 202 is marked as D. To improve isolation between the feeds it may be useful to ensure that electromagnetic radiation propagating in feed 206a and reflected from the inner wall of cavity 202 (e.g., ray 240) finds its way towards the other feed 206b. A mode having a local intensity maximum at the meeting point of ray 240 with the inner wall may suffer from strong coupling between the feeds, but other modes may enjoy improved inter-feed isolation. To estimate a proper distance X between the feeds, one may use Snell's low, according to which

sin θ₁\sqrt{∈_{waveguide}}=sin θ₂\sqrt{∈_{conduit}},

wherein θ₁=90°−α; and θ₂ is defined in FIG. 2.

Using basic trigonometry it is easily verified that

$$tan {θ}_{2} = \frac{X / 2}{D} ;$$

and after using Snell's law and rearranging, it may be shown that

$$X = \sqrt{\frac{n cos α}{1 - {( n cos α )}^{2}}} .$$

Wherein n=\sqrt{∈_{waveguide}/∈_{conduit}}.

Thus, in some embodiments, the distance between the feeds X and the inclination angle α obey the above relationship.

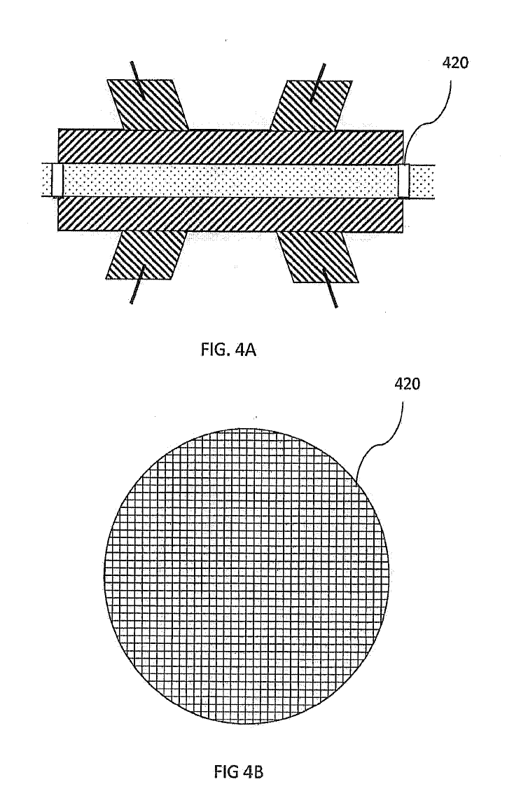

In some embodiments, for example, the embodiment shown schematically in FIGS. 4A and 4B, the apparatus may include an attenuator (420) that attenuates the intensity of the electrical field exiting from the cavity. While the field intensities used for investigating the material in the cavity may be low, such that no health or regulatory issues may arise from leakage of radiation from the cavity, it may be beneficial to attenuate the field outside the cavity, to decrease sensitivity of the measurements to changes in the electrical characteristics away from the cavity. Such changes may be caused, for example, by anything that may interact with the field along the conduit, in which the fluid flows to the cavity or from the cavity. If the apparatus is to be installed in a field, where other operations may be carried out, undefined changes in the electrical environment may be expected, and if these interact with the field, they may change the results of measurements taken inside the cavity. If, however, the field intensity outside the cavity is small, the influence of events outside the cavity on the measurement results is also small. Thus, in some embodiments, the field intensity after the attenuator is at least 100 times, in some embodiments at least 1000 times, smaller than inside the cavity (for example, at the cavity center, or the average across the entire cavity). In some embodiments, the attenuator may interfere with the material flow. For example, attenuator 420, shown in FIG. 4A, may include a metallic net, as shown in FIG. 4B, covering, or at least partially covering, the material inlet into the cavity and/or a metallic net covering the material outlet from the cavity. In some embodiments, the net may include square apertures about λ/10 long, where λ is the wavelength of the highest frequency used. For example, if the frequency range used for investigating the material is 1-6 GHz, and the dielectric constant of the material filling the cavity is 4, then λ is

$$\frac{3 \cdot {10}^{10} cm \text{/} sec}{\sqrt{4} \cdot 6 \cdot {10}^{9} Hz} = {2.5}_{c m} ,$$

and the net may include square aperture having dimensions of 2.5ₘₘ×2.5ₘₘ. Less dense nets may also be used, with smaller attenuation power, for example, less dense nets may allow leakage of radiation of high frequencies. Measurements taken by these high frequencies may be influenced by the field outside the cavity. In some embodiments, where the investigation of the material includes comparison between dielectric response of the cavity to dielectric responses measured before, in the presence of material of known properties, such comparisons will be less accurate, since the comparison will be between two measurements taken under different conditions.

In some exemplary embodiments of the disclosure, the cavity is open on at least one distal end. For example, according to one aspect, the cavity is substantially cylindrically-shaped, which is open on one or both distal ends.

As a result of the one or more openings, a portion of the electric field can be observed beyond the boundaries of the cavity. In some cases, this can lead to a change in the measurements obtained. Thus, the attenuator 420 serves to attenuate the electric field such less than 1% of the electric field will exit the cavity.

As will now be discussed below, the attenuator 420 may include an RF reflective attenuating conduit portion and a dielectric attenuating conduit portion. In this arrangement, the dielectric attenuating conduit portion is an extension of or is attached to the RF reflective attenuating conduit portion.

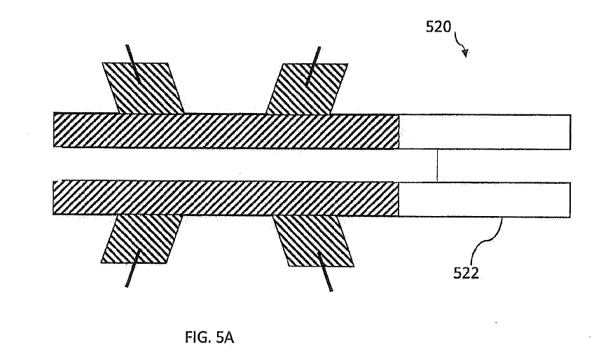

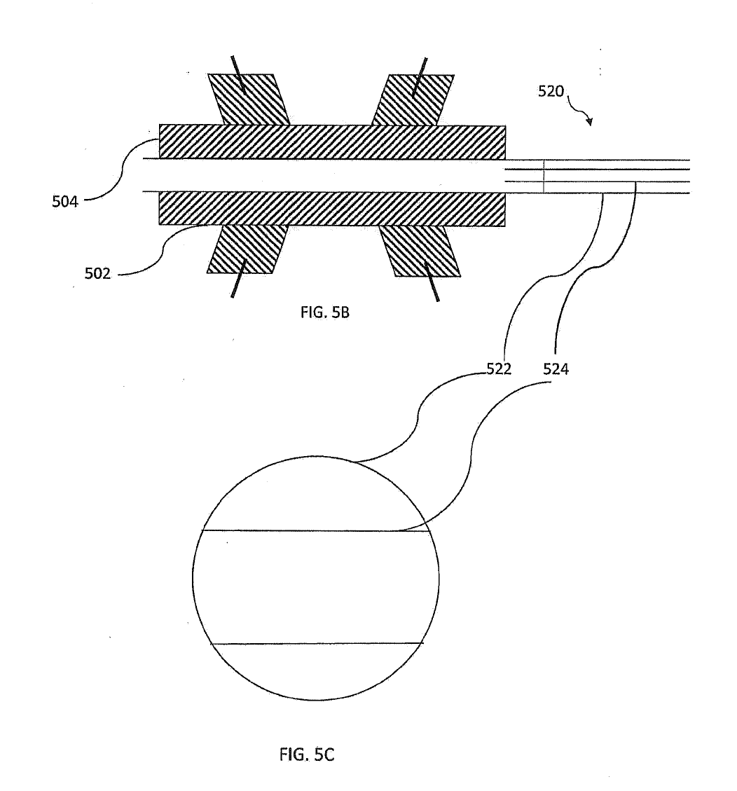

In some embodiments, for example, in the embodiment schematically depicted in FIGS. 5A and 5B, an attenuator 520 may include an attenuating conduit portion 522. Conduit portion 522 may be made of RF reflective material, e.g., may be metallic. The inner diameter of conduit portion 522 may be similar to that of dielectric conduit 504, such that flow of material will not be influenced, or be influenced only nominally, by the diameter difference between conduit 504 and conduit 522. For example, in some embodiments, the inner diameters of dielectric conduit 504 and metallic conduit 522 may be the same within a tolerance of 1 mm, 0.5 mm, or 0.1 mm. In FIG. 5A the metallic attenuating conduit portion 522 may be long enough to allow all the energy exiting from cavity 502 to absorb in the material flowing along attenuating conduit portion. In some embodiments, the inner diameter of attenuating conduit portion 522 may be the same as the inner diameter of dielectric conduit 504.

In FIG. 5B, attenuator 520 includes, further to a metallic attenuating conduit portion 522, partitions 524 going along attenuating conduit portion 522, to practically divide it into a plurality of waveguides extending parallel to each other. Partitions 524 may filter out radiation at frequencies that are below the cutoff frequency of the waveguides formed by the partitions. FIG. 5C is a diagrammatic illustration of a front view of attenuator 520 of FIG. 5B.

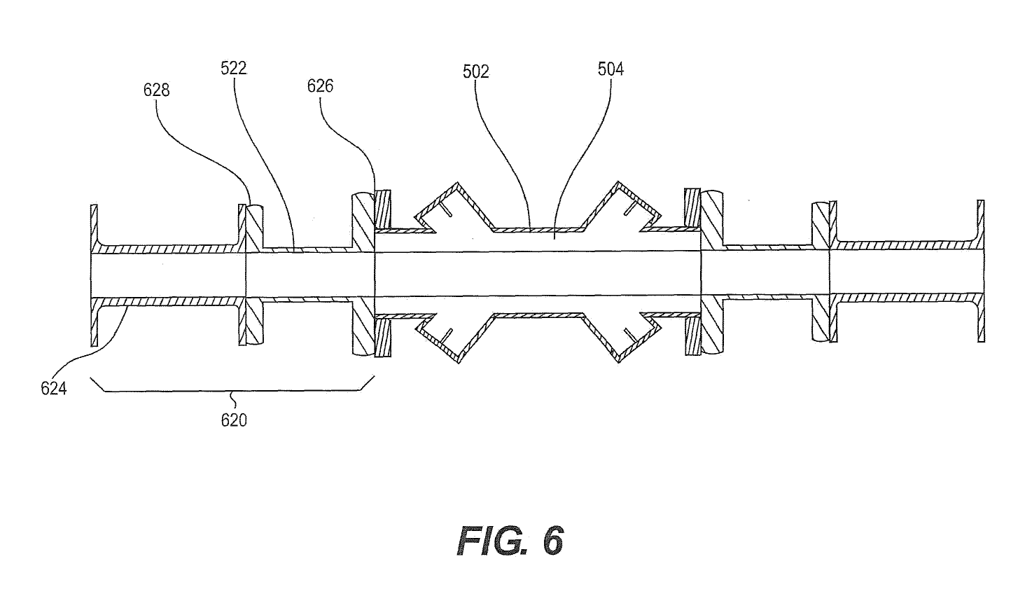

In some embodiments, for example, in an embodiment schematically depicted in FIG. 6, an attenuator (620) may include a metallic attenuating conduit portion 522, and further, a dielectric attenuating conduit portion 624. Metallic attenuating conduit portion 522 may be attached to cavity 502, for example, with flange 626. At its other end, metallic attenuating conduit portion 522 may be attached to dielectric attenuating conduit portion 624, for example, with flange 628. In some embodiments, the inner diameter of metallic attenuating conduit portion 522 and the inner diameter of dielectric attenuating conduit portion 624 are substantially the same, to avoid influencing the flow of the material to be investigated, as discussed above in regard of the inner diameters of dielectric conduit 504 and attenuating conduit portion 522. Accordingly, in some embodiments, the flow of material through conduit 504 and conduit portions 522 and 624 is smooth.

With this arrangement, the attenuating conduit portion is configured such that the field intensity at the distal ends of each dielectric attenuating conduit portion farthest from the metallic conduit portion is less than 1% of the field intensity existing inside the cavity.

In some embodiments, an attenuator may be provided only at one end of the cavity. In some embodiments (e.g., as shown in FIG. 6), both ends of the cavity (e.g., both fluid inlet and fluid outlet) may have attenuators. In some embodiments, the attenuator is configured and positioned with respect to the cavity so as to not interfere with the flow of material through the cavity, i.e., in a manner that will avoid interfering with the flow of material.

Apparatus 100 may further include detector 120. Detector 120 may be configured to detect parameters indicative of electrical response of the cavity to RF radiation fed to the cavity via feeds 106. Such parameters may be termed herein electrical response indicators. The detector may form part of a network analyzer, for example, a vector network analyzer. The parameters detected by the detector may include, for example, network parameters (e.g., s parameters, z parameters, input impedance z₀), their magnitudes and/or phases, or any other parameter that may be indicative to relationships between electromagnetic waves going into the cavity and out of it, for example, Γ parameters (scalar or complex). In some embodiments, both magnitudes and phases of the parameters may be detected by detector 120. In some embodiments, investigation may be carried out using magnitudes alone. In some embodiments, investigation may be carried out using phases alone.

In some embodiments, apparatus 100 may further include a source of radio frequency (RF) (microwave) radiation 115. The source may include any variable frequency signal generator, e.g., a direct digital synthesizer (DDS). The source may be configured to supply RF energy in the frequency range used for investigating the material, as this is discussed above. In some embodiments, one source is configured to feed all the feeds. For example, the source may be switched to feed one feed at a time. In some embodiments, the output of a single source may be divided to two or more of the feeds. In some embodiments, each feed may have a source of its own. The same may be true regarding the detector: in some embodiments, a single detector may detect signals going through and from the cavity through one source at a time, and be switched between the different feeds. In some embodiments, each feed may be connected to its own detector. It is noted, that in some embodiments the source and detectors may be integrated together, e.g., like in a network analyzer. The detector and the source may be configured to deal with (i.e., generate and detect) signals at a plurality of frequencies, for example, it may generate frequencies at any one of the above-mentioned sub-ranges of the microwave or RF range, at a controlled manner. As mentioned earlier, the more frequencies that may be used for excitation of field patterns in the cavity, the accuracy of the apparatus may improve. The patterns and/or predetermined patterns may be applied through only one feed, a plurality of feeds one at a time, a plurality of feeds at the same time (e.g., at the same frequency and at controlled phase difference between the feeds), or all of the feeds.

Apparatus 100 may include processor 130. The processor may be configured to determine the value of the property based on the parameters detected by the detector. Further, in some embodiments, processor 130 may control source 115. For example, processor 130 may control which frequency is generated at each instance. In some embodiments, processor 130 may be accessible to a memory storing some pre-defined frequency ranges and control source 115 to generate signals in these frequency ranges only.

The processor 130 may be a general purpose processor or may be part of an application specific integrated circuit (ASIC). The processor 130 may also be a microprocessor, a microcomputer, a processor chip, a controller, a microcontroller, a digital signal processor (DSP), a state machine, or a programmable logic device. The processor 130 may also be a logical circuit, including a programmable gate array (PGA) such as a field programmable gate array (FPGA), or another type of circuit that includes discrete gate and/or transistor logic. The processor 130 may be a central processing unit (CPU), a graphics processing unit (GPU), or both. Additionally, the processor 130 described herein may include multiple processors, parallel processors, or both. Multiple processors may be included in, or coupled to, a single device or multiple devices. Further, the processor 130 can be used in supporting a virtual processing environment.

In some embodiments, processor 130 may determine the value of the property based on parameters measured with materials having known properties. For example, to determine the volume fraction of water in an unknown emulsion of water in oil, the s parameters measured from the cavity with the unknown emulsion may be compared with s parameters measured from the cavity with known emulsions. The measurement of the known emulsions may be termed a training stage. The training stage may take place at a training apparatus. The training apparatus may be the very same apparatus where the unknown emulsion is treated (testing apparatus). In some embodiments, the training apparatus and testing apparatus may be different apparatuses of similar construction, i.e., duplicates. For example, the two apparatuses may have cavities of the same size, feeds arranged in the same manner, and generally, their detectors may be known to detect the same values of the parameters when the same emulsions flow in them. During training, spectrums of electrical response indicators (e.g., s parameters) vs. frequency may be obtained.

In some embodiments, the radiation may be applied through each feed at a time, and each feed may have its own spectrums. For example, in a four-feed apparatus, feed #1 may be associated with four spectrums: S11, S21, S31, and S41, each as a function of frequency. More generally, in an n-feed apparatus, feed #i may be associated with n different spectrums: Sji wherein j may have any integer value between 1 and n. It is noted that while in many prior art methods the non-diagonal members of the S matrix (i.e. Sⱼᵢ where i≠j) are the source of information, in many embodiments disclosed herein the main source of information are the diagonal members of the S matrix (i.e. Sⱼᵢ where i=j), while in some embodiments the non-diagonal members may be neglected, or even not measured at first place. In some embodiments, radiation may be applied through two or more feeds at overlapping time units, and Γ parameters may be measured. Γ parameters may also be associated each with a feed.

Determining the property of a material under investigation, also referred to as testing material, may include comparing spectrums measured with the testing material with spectrums of the same electrical response indicator obtained during the training stage with various training materials. The property of the material under investigation may be determined as the property of the training material that had a spectrum most similar to that measured in the testing stage. In this context, similarity may be determined by any known mathematical method, for example, kernel method, such as support vector machine.

In some embodiments, processor 130 may be configured to re-arrange the spectrums before comparison. For example, the processor may be configured to calculate one or more spectrums of combined parameters. For example, a new parameter may be defined, and spectrums of this new parameter may be compared in order to determine the property of the test material. That is, the processor 130 can combine parameters measured by the detector in order to obtain combined parameters. In doing so, the processor can then determine the value of the property based on the combined parameters.

In exemplary embodiments of the disclosure, each of the combined parameters is associated with a feed. One example of a combined parameter is a dissipation ratio (DR), which, in some embodiments, may be defined for each feed according to the following equation:

$${DR}_{i} = 1 - {\sum }_{j = 1}^{n} {\vert {S}_{ji} \vert}^{2}$$

The dissipation ratio may be indicative to that portion of the incident energy fed to the cavity via feed i that was dissipated in the cavity. This parameter is useful in selecting frequencies for heating, but it was surprisingly found to be useful also for determining properties of materials.

Another example of a combined parameter is the difference between values of a parameter measured at different times. For example, a reflection coefficient Sᵢᵢ may be measured at a given frequency at different times. The values measured at different times may be subtracted one from the other, and the difference may become a combined parameter. Such measurements and subtractions may be carried out at a plurality of frequencies (e.g., 50 frequencies) taken from an unknown sample, and the differences obtained at the various frequencies may be considered a spectrum. Such a spectrum may be compared with similar spectrums obtained from test samples of known properties, to estimate the properties of the unknown sample. The properties may include, for example, composition (e.g., water cut, gas content) gas flow rate and/or liquid flow rate.

In some embodiments, the variety of field patterns excited in the cavity may be further enriched by simultaneous irradiation through two or more feeds at controlled phase difference between them. Different phase differences may excite in the cavity different field patterns even at the same frequency. In such cases, the s parameters themselves are not measurable, and gamma parameters may be more useful. For example, the absolute values of the gamma parameter measured at a feed may be used as an electrical response indicator, the spectrum of which may be compared between the test material and the training material. The scalar |Γ|² parameter, may be defined for each feed i as

$${\vert {Γ}_{i} \vert}^{2} = \frac{{P}_{i}^{back}}{{P}_{i}^{forward}}$$

Wherein Pᵢ^{forward} stands for the power measured to go towards the cavity at feed i, and Pᵢ^{back} is the power measured to get back from the cavity to feed i. In some embodiments, it may be advantageous to combine parameters such that one parameter may be associated with each feed, so the spectrums will include one spectrum per feed, and each spectrum may include one value per frequency. In some embodiments, however, the combined parameter may be a single parameter based on information relating to all the feeds. For example, a feed-independent dissipation ratio may be defined, and used for determination of properties of the test material. Such a dissipation ratio may be given by the following equation:

$$DR = \frac{{\sum }_{i = 1}^{n} {\vert {a}_{i} \vert}^{2} {\vert {Γ}_{i} \vert}^{2}}{{\sum }_{i = 1}^{n} {\vert {a}_{i} \vert}^{2}}$$

Wherein n is the number of feeds, |Γᵢ|² was defined above, and αᵢ is the ratio between the waves measured in the forward (i.e. to the cavity) at feed i and the waves measured in the forward direction in feed 1, so α₁=1.

In some embodiments, processor 130 may be configured to determine the value of the property of the test material using kernel methods, for example, support vector machine. Accordingly, in some embodiments, analysis of the measured spectrums may include associating an index to an RF spectrum measured from the object whose property is to be determined, and determining a property of the object based on the index. The property may be determined from the index using a predetermined association, for example, in a form of a lookup table, between index values and properties.

In some embodiments, the property index (P) may be calculated based on kernels (k), each kernel being a value of a kernel function (K). A kernel function (K(\vec{X}, \vec{V})) may be a mathematical function that fits a number to a pair of vectors. The kernel function should have some additional properties, as known in the field of structured learning. In some embodiments, each spectrum is represented as a vector. For example, a spectrum including 100 points (each point being association between a frequency or other excitation setup and a value of an electrical response parameter) may be represented by a 100-dimensional vector. The kernel function may depend on the dot product of the measured spectrum \vec{X} by the reference spectrum \vec{V}, which may be a spectrum of an object with a known property, measured in the training stage. Each reference spectrum \vec{V} may be associated with a property indicator, y, which indicates which property the reference object is known to have. For example, the indicator may have a value of −1 for one property (e.g., water content smaller than 5%) and +1 for another property (e.g., water content of 5% or more). In some embodiments, each reference spectrum may be associated with a weight α. In some embodiments, the property index (P) to be associated with an object, from which a spectrum \vec{X} was measured, may be given by the equation:

$$P = {\sum }_{j} {α}_{j} {y}_{j} K ( \vec{X} \cdot {\vec{V}}_{j} )$$

Thus, evaluating the property index (P) may include determining values of a kernel function of the measured spectrum and a plurality of reference spectrums to obtain a plurality of kernels; multiplying each kernel by the weight of the corresponding reference spectrum and by the group indicator of the reference spectrum, to obtain multiplicative products αⱼyⱼK(\vec{X}·\vec{V}ⱼ), and summing the multiplicative products to obtain the index.
To obtain the reference spectrums, their weights, and their property indicators, measurements may be made on reference objects having known properties during the training stage. Each reference spectrum may be associated with a property indicator according to the property of the reference object. Each reference spectrum may be further associated with a weight, indicative of the importance of the reference spectrum in distinguishing between objects having the different properties.

Obtaining the reference spectrums and analyzing the data using them may be accomplished in a known kernel method, e.g., support vector machines (SVM), Gaussian processes, Fisher's linear discriminant analysis (LDA), principal components analysis (PCA), canonical correlation analysis, ridge regression, spectral clustering, linear adaptive filters, etc.

FIG. 3 is a flow chart of a method 300 of determining a value of a property of a material that flows in a conduit inside a microwave cavity according to some embodiments of the invention. Method 300 may include a step 302 of exciting multiple modes in the cavity. The excitation may be through a number of feeds, as discussed above. For example, one (or more) of the feeds may include a radiating element outside the cavity and a waveguide configured to guide waves from the radiating element to the cavity. Exciting the multiple modes may include transmitting into the cavity microwave radiation at different frequencies, and if multiple feeds are provided, exciting the multiple modes may include transmitting the waves through different ones of the feeds. Generally, it may be said that the different modes may be excited by transmitting to the cavity microwave radiation at different excitation setups, wherein each excitation setup is defined by the transmitting feed and by the transmitted frequency. In some embodiments, when waves are transmitted simultaneously through a plurality of feeds, at a common frequency, and at controlled phase differences between the feeds, the excitation setup may be further defined by the phase differences. If other parameters that may affect the field pattern excited in the cavity are also controllable by apparatus 100, the excitation setups may be further defined by them.

In exemplary embodiments of the disclosure, exciting a number of modes is by applying to the cavity RF radiation at a plurality of excitation setups. In some embodiments, each two of the excitation setups differ from one another in at least one of a frequency or a feed, through which RF radiation is fed to the cavity to obtain the excitation. By applying excitation to different feeds, through their respective ports, and at different frequencies, excitation of various modes can be achieved.

In some embodiments, excitation of the modes includes exciting a number of modes that is larger than the number of the feeds. For example, if the feeds are inclined as described above, and each feed excites in the cavity one mode of each type (e.g., TE, TM, and quasi-TEM), the number of modes may sometimes be three times larger than the number of feeds.

Method 300 may further include step 304 of detecting parameters indicative of electrical response of the cavity to the excitation of the modes in the cavity. As discussed above, such parameters may include network parameters (e.g., s parameters), gamma parameters, or any other electrical response indicator. It is noted that in some embodiments, for example, where the various feeds are decoupled, parameters indicative of radiation transfer from one feed to another (e.g., Sᵢⱼ, i≠j) may be less informative than parameters indicative of reflections back to the emitting feeds (e.g., Sᵢᵢ or Γ parameters also known as gamma parameters). Accordingly, in some embodiments, the parameters indicative of electrical response of the cavity to RF radiation fed to the cavity may include a ratio between power measured to go towards the cavity at a given feed, and power measured to get back from the cavity towards the given feed. If the feeds emit each at a time, this ratio may be a diagonal s parameter; if the feeds emit at overlapping time periods, this ratio may be a Γ parameter. In some embodiments, only magnitudes of the S or gamma parameters are considered, while in other embodiments, the phases of the parameters are also considered.

Finally, method 300 may include step 306 of determining the value of the property based on the detected parameters. This may include, in some embodiments, comparing electrical response indicators (either as measured, or after further processing, e.g., combination as discussed above) with values obtained from reference materials during a training state. In some embodiments, the comparison may include usage of by a kernel method, such as support vector machine.

Some embodiments of the invention may include RF-based flow rate measurements. The measured flow rate may be of a foreign body flowing within a material. The material itself may be flowing or stationary. In the latter case, the foreign body may be moved in the stationary material, for example, by ultrasonic waves. The foreign body may have a dielectric constant different from that of the material, so it reflects RF radiation to a different extent. Some examples of foreign bodies may include gas bubbles in a liquid material, oil droplets in water, solids in liquids, etc. In some embodiments, measuring the flow rate may include comparing frequencies of signals transmitted at one point along the flow path to frequencies received at another point down the flow path. Due to the Doppler Effect, the frequency of the received signal may be shifted in respect to the frequency of the transmitted signal by a degree indicative of the flow rate.

In some embodiments, the measurements may take place inside a microwave cavity. The microwave cavity may include metallic walls encasing a dielectric conduit, along which the material may flow. Since tangential electric field components tend to vanish in the vicinity of metallic walls, such as walls of microwave cavities, foreign bodies moving near to the walls of the cavity may be hard to detect. The dielectric conduit may limit the flow of the material to regions where the distance from the wall is large enough to ensure that the electrical field does not vanish within the material due to closeness to the metallic wall. Thus, in some embodiments, the thickness of the conduit is at least ¼ a wavelength of the RF radiation used for measuring the flow rate. The said wavelength may be a wavelength inside the dielectric material constituting the conduit. In some embodiments, the conduit may be covered by Teflon material which may facilitate detection of foreign bodies moving near the conduit walls.

In some embodiments, the signals are transmitted through a single radiating element at a time. In some embodiments, the signals are transmitted through multiple radiating elements at overlapping time periods and at the same frequency. The multiple transmitting radiating elements may be positioned at different points along a perimeter of the microwave cavity, and at a common distance from an end of the flow path of the foreign body within the conduit. In some embodiments, the signals may be received by two or more radiating elements.

The comparison may be of the signal, or of the electrical response of the cavity to the signal. The dielectric response may be expressed, for example, by the network parameters of the cavity with the material and foreign body flowing therein. In some embodiments, values of network parameters may be used for the measurements. For example, the above-mentioned frequency shift may be detected as a time varying phase shift in a transfer parameter (Sij, i#j) parameter. More generally, when a foreign body is moving (e.g., flowing), the electrical response of the cavity with the moving foreign body will vary over time, and this variation may be used to estimate the flow rate.

In some embodiments, measurements may be taken at a plurality of frequencies. This may be advantageous in that different frequencies may excite in the flowing material different modes. The sensitivity of the measurement may depend on the field intensity at the immediate location of the foreign body. Since different modes may have field maximums at different locations, different frequencies may allow sensitive measurements of bodies that flow at different portions of the conduit. Thus measurements taken at a plurality of frequencies may be sensitive to foreign body motion at many different portions of the conduit, and in some embodiments, practically everywhere within the conduit between the transmitter and the receiver. In some embodiments, differing modes or differing field patterns may be excited with a single frequency. For example, the same frequency may be emitted through differing radiating elements, resulting in the excitation of differing field patterns in the conduit. In another example, two or more of the radiating elements may concurrently radiate at the same frequency and at differing phase differences between them, resulting in excitation of a plurality of field patterns, and thus increase the sensitivity of the measurement method. The field patterns may include two or more field patterns that are significantly different from each other. In some embodiments, two field patterns may be considered significantly different from each other if a position with a low electric field (e.g., smaller than 20% of the maximal electric field) of the first field pattern has a high electric field (e.g., larger than 50% of the maximal electric field) within the second field pattern.

In some embodiments, measurements may be carried out based only on signals having intensity above a threshold. The threshold may be set based on the noise known to exist in the system. For example, in some embodiments, only signals having a signal to noise ratio of at least 2, at least 3, at least 4, etc., may be taken into consideration. In some embodiments, the noise level may be detected during operation, and the threshold may be adjusted online to noise existing in the system at every instance. For example, the noise may change from time to time and the threshold may be automatically adjusted accordingly. Such automatic threshold adjustment may be facilitated by receiving RF radiation from a region within the conduit, which is not accessible to foreign bodies, or much less accessible than most other portions of the conduit. Thus, every signal received from such a region may be treated as noise, and the threshold may be automatically adjusted based on readings from such a non accessible region. Such automatic threshold may also be determined according to noise level within a Doppler frequency (up to 2v/λ) in which there are no signals from foreign bodies due, for example, to the limit present on the maximal flow speed of the foreign body which is typically the flow rate of the material in which the foreign body flows. This may be possible since the flow rate is proportional to the maximal Doppler frequency induced by the foreign body.

In some embodiments, the maximal size and the minimal flow velocity of the foreign body may be expected to have are known, and, automated threshold adjustments may be set by comparing Doppler signal reflections at times before or after the foreign object has passed through the conduit, and during the passing of the foreign object between the radiating elements. The signal to noise ratio to be crossed by a signal may be set before measurements begin. This ratio may be, for example between 2 and 4. In general, the larger is the ratio—smaller number of signals is taken into account, and more false negative and less false positive readings may be expected.

In some embodiments, the amount and volume of the foreign objects may be estimated by measuring the strength of a Doppler signal. The Doppler signal may be correlated to the amount and volume by a proportionality constant. In case the foreign object is to be detected on the background of a non-homogenous flow, the Doppler signal from each location in space may be calculated separately thereby providing the ability to perform detection by comparing the Doppler signal from each small spatial volume to the Doppler signal from its neighboring volumes. Calculation of signals and their origin in space may be accomplished by coherently summing reflections from different frequencies and radiating elements with appropriate weights, such that each weight set emphasizes contributions to the Doppler signals from a different location in space.
In some embodiments, an apparatus (e.g., apparatus 100) for detecting phase-composition of a multi-phase material (e.g., crude oil) may include RF-based flow rate detection (e.g., by measuring Doppler signals). In some embodiments, it may be assumed that the different phases of the multi-phase material (e.g., water, gas and oil of a crude oil) are flowing in unison and thus the flow rate of the multi-phase material is identical or similar to the flow rate of the gas. The flow rate of the multi-phase material may be detected by detecting a flow rate of a gas flowing within the multi-phase material (e.g., crude oil). In some embodiments, the gas may be treated as the foreign object to be detected (e.g., by Doppler means as discussed above).

In some embodiments, Doppler detection (for example: as discussed above) may be used for detecting foreign object in a flowing material: e.g., for detecting undesired objects flowing in the material—for example: in food industry—it may be desired to detect foreign objects in a food being processed (e.g., flowing milk, juices, crème etc.). In some embodiments, the size of the detected foreign object may be in the range of: mm3 (e.g., metal balls or glass/plastic beads having diameter of 2-6 mm, e.g., 3 mm). In some embodiments, once the foreign object is detected—an alert may be sent to the operator at the factory such that the foreign object may be removed.

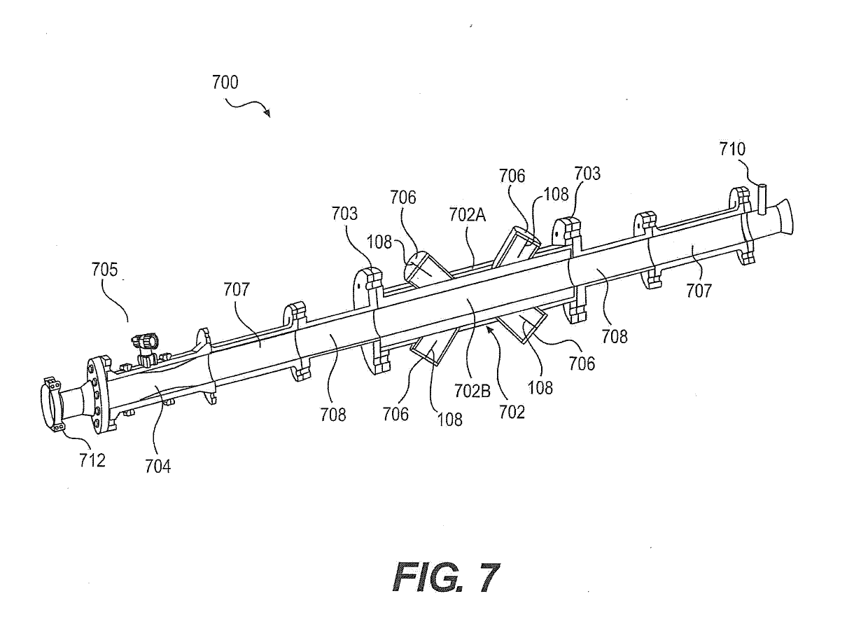

FIG. 7 is a diagrammatic illustration of an apparatus 700 according to some embodiments of the present disclosure. Apparatus 700, may be a multi-phase flow meter. Apparatus 700 may operate similarly to apparatus 100. The multi-phase flow meter 700 includes a resonance cavity 702, also referred to herein as a microwave cavity, a venturi tube 704, differential pressure (DP) transmitter 705, dielectric attenuating conduit portions 707, formed as plastic end members, metallic attenuating conduit portions 708, a sensor assembly 710, and interfaces (not shown) to a local processor, e.g., as shown in FIG. 1A (processor 130).

The resonance cavity 702 includes a metallic outer piping section 702a, a dielectric inner piping section 702b (also referred to herein as a conduit), flanges 703, and waveguides 706. In some embodiments, the metallic outer piping section 702a has an internal diameter of 90 mm and a length of 380 mm. In some embodiments, the dielectric internal piping section 702b has an external diameter of 90 mm and an internal diameter of 52.5 mm. In some embodiments, the dielectric internal piping is made of PTFE (Teflon) and has a dielectric constant of about 2.2. It is noted that any suitable diameters, lengths and materials may be used. For example, the outer piping section 702a may have internal diameter of between 40 mm and 200 mm, and length of between 180 mm and 800 mm. The dielectric piping section may have an external diameter equal to the internal diameter of the outer piping section, and an internal diameter of between about 5 mm and about 65% of the outer diameter. The internal piping may be made of materials having dielectric constants of, for example, from about 1 to about 10.

In some embodiments, the waveguides 706 include four metallic tubes approximately 49.2 mm from the center along the pipe at a 40° angle, having an internal diameter of 52.5 mm and a length of 64 mm. In some embodiments, the waveguides 706 are filled with alumina Al₂O₃ having a density of at least 3.85 gr/cm³, and dielectric constant of 9.5. It is noted that any suitable diameter, length, angle, and composition of the waveguides 706 may be used. For example, the internal diameter of the waveguides (formed as metallic tubes 706) may be substantially the same as the internal diameter of the internal piping section 702b.

Flanges 703 may be formed from 180 mm diameter metal pipe sections; although, any suitable dimensions and material may be used.

The metallic attenuating conduit portions 708, are connected to each end of the resonance cavity 702. In exemplary embodiments, the metallic attenuating conduit portions have an internal diameter of 52.5 mm and a length of 150 mm; although, any suitable diameter and length may be employed. For example, the internal diameter of the metallic attenuating conduit portion 708 may be about the same as the internal diameter of the dielectric internal piping. On the ends of the metallic attenuating conduit portions 708 closest to the resonance cavity 702, a 180 mm in diameter metal pipe section is used as a flange, whereas on the ends of the blocking pipes farthest from the resonance cavity 702, a 128 mm in diameter metal pipe section is used as a flange. It is noted that any suitable dimensions and material may be used as flanges.

The dielectric attenuating conduit portions 707 may include 52.5 mm internal diameter fiberglass tubes having a length of 180 mm. In some embodiments, the dielectric attenuating conduit portions 707 are made from glass fabric reinforced composite material. A 128 mm diameter flange may be used to connect the dielectric attenuating conduit portions 707 to the metallic attenuating conduit portions, and a flange of similar diameter (e.g., 128 mm) may be used to connect one of the dielectric attenuating conduit portions 707 to the venturi tube 704. It is noted that any suitable dimensions may be used for the dielectric attenuating conduit portions 707 and the respective flanges. For example, the internal diameter of the dielectric attenuating conduit portions 707 may be substantially the same as the internal diameter of the dielectric internal piping. The length of the dielectric attenuating conduit may be longer than 180 mm, with longer conduits providing better attenuation. However, other requirements relating, for example, to the overall size of the apparatus, may dictate using a conduit of only 180 mm in length, or even shorter.

The venturi tube 704, in some embodiments, has an internal diameter of 1.5 inches (38.1 mm) and may be connected to multi-phase flow meter 700 by a weld-neck flange that expands to the internal diameter of the dielectric internal piping. It is noted that any suitable dimensions may be used. The venturi tube 704 includes a high pressure connection and a low pressure connection.

The differential pressure transmitter 705 includes a transmitter able to transmit results of differential pressure measurements wirelessly to one or more computers, servers, or other remote devices. The differential pressure transmitter 705 may transmit results of differential pressure measurements obtained at the high pressure connection and the low pressure connection of the venturi tube 704.

The waveguides 706 may include in one embodiment 50 ohm N-type RF connectors connected to 50 ohm RF cables. The RF cables may then be connected to and RF-matrix, which may then be connected to an analyzer, such as a vector network analyzer, which in turn may be connected to a controller or processor. An exemplary analyzer is the Agilent Technologies E5071C or N7018A. The vector network analyzer may be adapted to measure RF parameters according to wide-band signatures and/or time variance, as will be discussed in greater detail below.

The sensor assembly 710 may include one or more sensors adapted to measure pressure, temperature, and/or velocity of material flowing through the microwave cavity 702. This data may then be transmitted to a central server or other processing device via a suitable communications link, as will be discussed below.

In some embodiments, the apparatus 700 may be adapted to connect to external piping using, for example, two inch Grayloc™/DIN interface connectors, one of which is shown as part 712. Suitable flange connections between the interfaces 712 may also be employed.

For its resistance to corrosion properties, one or more of the previously discussed elements may be made of stainless steel, brass, or other suitable material able to withstand crude oil, corrosive substances, and solvents.

The temperature sensor of sensor assembly 710 may be positioned in or near the flow of material and may be adapted to measure the temperature of the material flowing in microwave cavity 702. The pressure sensor of sensor assembly 710 may be adapted to measure the pressure of the flowing material. If two pressure sensors are employed, a differential between the first pressure sensor and the second pressure sensor may be obtained in order to detect an increase or a decrease in the pressure of the flowing material. The velocity sensor of sensor assembly 710 may be adapted to measure the flow of material at two different points in time, to provide information as to the flow rate, e.g., as to the flow rate of the liquid. Optionally, a volume sensor may be included in sensor assembly 710, and detect the volume of material flowing. The readings obtained from the various sensors (e.g., pressure, temperature, volume, and/or velocity sensors) may be used to determine values characterizing the material. For example, during the training, spectra may be taken from samples in different temperatures, and different estimators may be created for each temperature. Then, in the estimation stage, the temperature measurements taken by the temperature sensor of assembly 710 may be used to tell which estimator is to be used for estimating the properties of the sample taken. The sensors may be able to transmit measurements wirelessly, or via a hardwired connection, to one or more computers, servers, or other remote devices.

Multi-phase flow meter 700 may be stationed at a site, for example, at an oil or gas well, or at another site that properties of a flow need to be measured and/or determined. A processor for analyzing the measurements results may be provided remotely from the site, in for example, a server that receives data and measurements from one or more sensors located at the site, via a communications network. In this regard, the one or more sensors may be located in or adjacent, for example, the oil or gas well. The one or more sensors may include, for example, the sensors identified above.

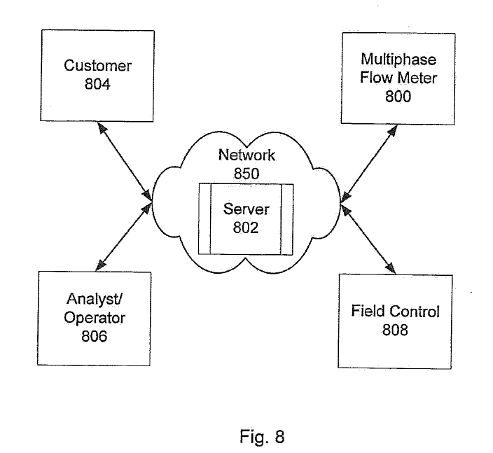

FIG. 8 is an exemplary architecture within which a multi-phase flow meter may be used, according to some embodiments of the present disclosure. The multi-phase flow meter 800 is connected to a server 802 by network 850. Server 802 may be internal to network 850 (as illustrated), or external to network 850. In some embodiments, the network 850 is a cellular network. In other embodiments, the network 850 is a wireless local area network (WLAN), a global area network (GAN), local area network (LAN), wide area network (WAN), metropolitan area network (MAN), global system for mobile (GSM) network, code division multiple access network (CDMA), public switched telephone network (PSTN), packet switched network, mobile network, Bluetooth compatible network, near field communication (NFC), a hard wired network, a wireless network, a landline network, Zigbee, or a Wi-Fi network. Of course, it is understood that any network known by one of ordinary skill in the art may be employed.

The server 802 may be accessed through network 850 by, for example, SCADA (supervisory control and data acquisition) 804, analysts and operators 806, and field control 808.

SCADA 804 may be, for example, the data acquisition and control system of an oil well, to which multiphase flow meter (MPFM) 800 may send through network 850 some of the data it acquired, optionally, in real time. In some embodiments, communication between MPFM 800 and SCADA 804, as well as between any other two of SCADA 804, Analyst 806, Field Contorl 808 and MPFM 800, goes through the server. Communication to and from the server may be through network 850. In some embodiments, network 850 may include server 802. The data sent to the SCADA may include, for example, flow rate data, data relating to composition of the flowing material, etc. The SCADA may accordingly control, optionally through network 850, field control 808. In some embodiments, MPFM 800 may send instructions to field control 808 via network 850, without involving the SCADA in the process. The field control may be operable to control, for example oil pumping from the well, routing pumping products in the oil fields, etc. For example, if MPFM 800 finds out that the water content in the material is above a threshold, MPFM 800 may instruct intermitting the pumping, for example, for a predetermined period, to allow the water to settle. This may be done via the SCADA or directly via network 850.

Server 802 may receive from MPFM 800 on-line data on properties of the materials flowing through MPFM 800. In some embodiments, there may be several MPFM 800 units (e.g., if a field includes more than one well), and server 802 may receive data from all of them. In some embodiments, server 802 may receive samples of raw data, e.g., of measured s parameters at the different frequencies. Such data may be used for further analysis and study. Raw data not sent to server 802 may be stored on MPFM 800 for a short while, and then deleted to free space for new data coming in in real time. Sending all the raw data to the server may provide the possibility of further control and analysis, but may be omitted, for example, if communication lines are not available or too expensive to carry all that data.

Analyst/Operator 806 may provide data to the SCADA and/or to MPFM 800. For example, Analyst/Operator 806 may determine the threshold of water content mentioned above. In some embodiments, analyst/operator 806 may receive data samples from MPFM 800 (e.g., via server 802), to allow further analysis of the field production, for example, to estimate the overall production of the field, how the production is distributed over time, different productivities of different wells in the field, etc.

Each of SCADA 804, analysts and operators 806, and field control 808 can include stationary computer, a mobile computer, a personal computer (PC), a laptop computer, a tablet computer, a wireless smart phone, a personal digital assistant (PDA), a control system, or any other machine capable of executing a set of instructions (sequential or otherwise) that specify actions to be taken by that machine.

The server 802 may be connected to a database or other storage and/or memory devices with which data can be stored for later retrieval. The server 802, database, and or other components of the systems with which the system interacts may be implemented in a cloud-based environment. For example, the cloud-based environment may include a network of servers and web servers that provide processing and storage resources.

In this regard, the aforementioned components may possess the necessary hardware and software communications facilities necessary for bidirectional communications between the various components discussed. In this fashion, data measured and observed via the multi-phase flow meter 800 or any of its components can be transmitted in real-time to the server 802 via a cellular communications module or other wireless communications module, or any combination of hardware and/or software requirements known to one of ordinary skill in the art to facilitate the transmission and reception of data. Thus, the multi-phase flow meter 800 can transmit measured and observed data to the server 802, and/or can transmit an audio and/or visual alarm signal indicative of a foreign substance in conduit 104 (see FIG. 1A), e.g., deposits of wax on conduit walls. The specific alarm signal to be sent can be dependent upon the particular foreign substance detected. For example, upon detection of a foreign substance, a lookup in a table or a query in a database, for example, could be performed to determine which foreign substance is associated with what alarm signal. In this regard, operators of the system can decide what substances are associated with what alarm signals, and store these preferences using an appropriate interface, for example via customers 804, operators 806 and/or field control 808. Alarm signals can also be sent for other conditions as high flow rate (e.g., higher than a predetermined high flow threshold), low flow rate (e.g., lower than a predetermined low flow threshold), and conditions associated with pressure differential, temperature, etc. For example, an alarm signal can be transmitted if a low flow rate is detected, which could be the result of an obstruction in the multi-phase flow meter, an obstruction elsewhere in the well or supply lines. Additionally, a high flow rate alarm signal can be provided to warn of a potential lack of capacity situation. Similarly, alarm signals associated with pressure differential can warn of potentially undesirable situations before becoming critical. The alarm signals can also be specific to the type of condition observed as discussed, i.e., high flow rate, low flow rate, etc.

Additionally or alternatively, field personnel and/or customers can transmit any operational instructions to the multi-phase flow meter 800 via software or Internet applications, for example. For example, field personnel can transmit instructions to the multi-phase flow meter 800 that instruct excitation to be applied through different feeds and different frequencies, in order to obtain the desired modes of excitation. Of course, other instructions can be sent as would be known by one of ordinary skill in the art.

The customers 804 are able to view the data from the server by an appropriate interface or portal accessible from suitable devices, as appreciated by one of skill in the art, associated with the customers.

An algorithm may be employed to support the identification of flow speeds and/or compositions of the flowing material. In some embodiments, the algorithm is a Python™ based program. The algorithm includes an estimation module, as will be discussed below.

In order to estimate the property of the material, e.g., the flow rate Q_{L} of a liquid through a sensor or a flow rate of gas through the sensor Q_{A}, the RF feeds in the sensor may provide samples of the scattering parameters Sii, measuring the reflection coefficient to feed i when voltage is applied to the same feed i. In some embodiments, the RF feeds may also provide samples of the scattering parameters Sᵢⱼ measuring the transmission coefficient to feed i, when voltage is applied at feed j. An S parameter at frequency w is denoted by Sᵢⱼ(w) where i and j may be the same, to provide reflection coefficients, or different, to provide transmission coefficients. In some embodiments, wide band signatures may be analyzed to provide properties of the flowing material, such as flow rate and composition. In some embodiments, time variation may be used to provide the material properties. In some embodiments, both time variation and broad band signatures may be used.

The wide-band signature RF measurement may be used to find both composition of the material and flow rate of the material. The time variation measurement may also be used to determine composition and/or flow rate of the material.

With respect to wide-band signature measurement, N evenly spaced frequencies in the range [L; H], where [L; H] is wide and N is large may be used. Exemplary values may be N=10⁴, L=1.0 GHz, and H=6.5 GHz. For such a set of frequencies, W, and for a set of feeds P={1; 2; . . . ; K} a wide-band signature x may be defined by:

x={Sᵢⱼ(w)|w∈W;i,j∈P}

A wide-band signature is a complex vector of size |W| |P|². In some embodiments, the dimensionality of the input space X may be reduced using supervised on unsupervised learning to obtain a reduced input space Z.

Given X (or Z) and the associated values of the properties (e.g., the flow rates and/or the compositions), Support Vector Regression may be used to find suitable mapping between the input space and the properties.

Machine learning techniques (for example, support vector regression) may be used to generate an estimator configured to estimate material properties based on the input vectors. For example, the input vectors, measured with materials of known properties, may be stored in a database in association with the corresponding material properties. This database may be used to generate the estimator. To use the estimator, spectra may be measured from materials having unknown properties (e.g., S parameters of the cavity when a material of unknown composition flows in the cavity at unknown flow rate). The estimator may then operate on these spectra (optionally, on these spectra in reduced form) to estimate the properties of the materials. In one embodiment (referred to herein as time variation embodiment), the input for the machine learning techniques and to the estimators they generate may include the degree by which the measured S parameter values vary over time. In such embodiments, the number of frequencies (or, more generally, excitation setups) used may be limited by the number of measurements that may be taken during a single time period. The single time period may be short enough so that the distance that the material flows within the single time period is small in comparison to the distance between the feeds along the material flow path.

Time-variation measures may use a smaller set of frequencies F, (e.g., |F|=100), where each frequency is sampled several times along a given time interval T. Thus, dSᵢⱼ(w)/dt is evaluated for every frequency w, so that the dynamics of Sᵢⱼ can be correlated with flow rate.

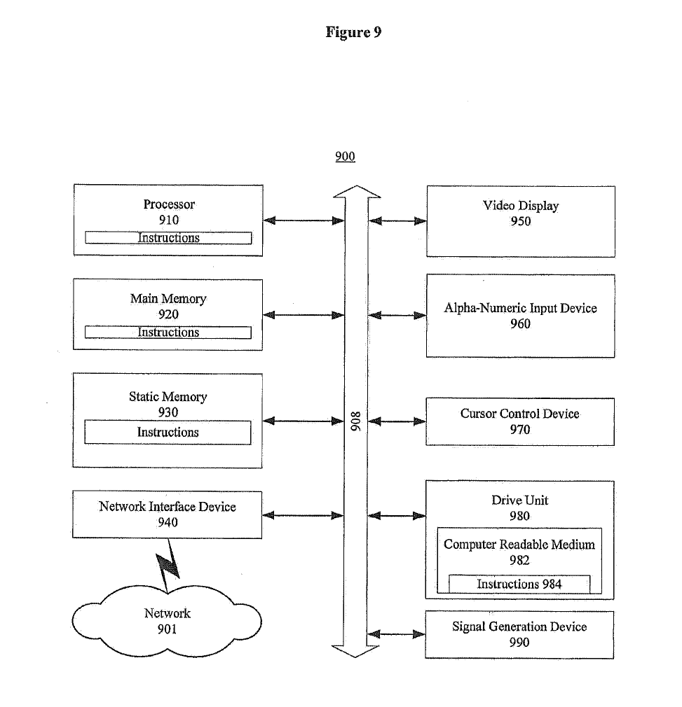

FIG. 9 is an illustrative block diagram of a general computer system 900, on which a method for determining a value of a property of a material according to some embodiments of the present disclosure can be implemented. The computer system 900 can include a set of instructions that can be executed to cause the computer system 900 to perform any one or more of the methods or computer based functions disclosed herein. The computer system 900 may operate as a standalone device or may be connected, for example, using a network 901, to other computer systems or peripheral devices.

The computer system 900 can also be implemented as or incorporated into various devices, such as a stationary computer, a mobile computer, a personal computer (PC), a laptop computer, a tablet computer, a wireless smart phone, a personal digital assistant (PDA), a control system, or any other machine capable of executing a set of instructions (sequential or otherwise) that specify actions to be taken by that machine. The computer system 900 can be incorporated as or in a particular device that in turn is in an integrated system that includes additional devices. In a particular embodiment, the computer system 900 can be implemented using electronic devices that provide voice, video or data communication. Further, while a single computer system 900 is illustrated, the term “system” shall also be taken to include any collection of systems or sub-systems that individually or jointly execute a set, or multiple sets, of instructions to perform one or more computer functions.

As illustrated in FIG. 9, the computer system 900 includes a processor 910. A processor for a computer system 900 is tangible and non-transitory. As used herein, the term “non-transitory” is to be interpreted not as an eternal characteristic of a state, but as a characteristic of a state that will last for a period of time. The term “non-transitory” specifically disavows fleeting characteristics such as characteristics of a particular carrier wave or signal or other forms that exist only transitorily in any place at any time. A processor is an article of manufacture and/or a machine component. A processor for a computer system 900 is configured to execute software instructions in order to perform functions as described in the various embodiments herein. A processor for a computer system 900 may be a general purpose processor or may be part of an application specific integrated circuit (ASIC). A processor for a computer system 900 may also be a microprocessor, a microcomputer, a processor chip, a controller, a microcontroller, a digital signal processor (DSP), a state machine, or a programmable logic device. A processor for a computer system 900 may also be a logical circuit, including a programmable gate array (PGA) such as a field programmable gate array (FPGA), or another type of circuit that includes discrete gate and/or transistor logic. A processor for a computer system 900 may be a central processing unit (CPU), a graphics processing unit (GPU), or both. Additionally, any processor described herein may include multiple processors, parallel processors, or both. Multiple processors may be included in, or coupled to, a single device or multiple devices.

Moreover, the computer system 900 may include a main memory 920 and a static memory 930 that can communicate with each other via a bus 908. Memories described herein are tangible storage mediums that can store data and executable instructions, and are non-transitory during the time instructions are stored therein. A memory described herein is an article of manufacture and/or machine component. Memories described herein may include computer-readable mediums from which data and executable instructions can be read by a computer. Memories as described herein may be random access memory (RAM), read only memory (ROM), flash memory, electrically programmable read only memory (EPROM), electrically erasable programmable read-only memory (EEPROM), registers, a hard disk, a removable disk, tape, compact disk read only memory (CD-ROM), digital versatile disk (DVD), floppy disk, blu-ray disk, or any other form of storage medium known in the art. Memories may be volatile or non-volatile, secure and/or encrypted, unsecure and/or unencrypted.

As shown, the computer system 900 may further include a display unit (e.g., video display unit 950), such as a liquid crystal display (LCD), an organic light emitting diode (OLED), a flat panel display, a solid state display, or a cathode ray tube (CRT). Additionally, the computer system 900 may include an input device 960, such as a keyboard/virtual keyboard or touch-sensitive input screen or speech input with speech recognition, and a cursor control device 970, such as a mouse or touch-sensitive input screen or pad. The computer system 900 can also include a disk drive unit 980, a signal generation device 990, such as a speaker or remote control, and a network interface device 940.

In a particular embodiment, as depicted in FIG. 9, the disk drive unit 980 may include a computer-readable medium 982 in which one or more sets of instructions 984, e.g. software, can be embedded. Sets of instructions 984 can be read from the computer-readable medium 982. Further, the instructions 984, when executed by a processor, can be used to perform one or more of the methods and processes as described herein. In a particular embodiment, the instructions 984 may reside completely, or at least partially, within the main memory 920, the static memory 930, and/or within the processor 910 during execution by the computer system 900.

In some embodiments, dedicated hardware implementations, such as application-specific integrated circuits (ASICs), programmable logic arrays and other hardware components, can be constructed to implement one or more of the methods described herein. One or more embodiments described herein may implement functions using two or more specific interconnected hardware modules or devices with related control and data signals that can be communicated between and through the modules. Accordingly, the present disclosure encompasses software, firmware, and hardware implementations. Everything in the present application should be interpreted as being implemented or implementable with software, hardware, or a combination of software and hardware.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing can be constructed to implement one or more of the methods or functionality as described herein, and a processor described herein may be used to support a virtual processing environment.

The present disclosure contemplates a computer-readable medium 982 that includes instructions 984 or receives and executes instructions 984 responsive to a propagated signal.

Accordingly, the present disclosure enables a method and apparatus for determining a value of a property of a material that flows in a conduit inside a microwave cavity.

For example, the processes contemplated herein include the determination of the water cut of crude oil, water liquid ratio (WLR), and/or gas volume fraction (GVF). The methods and apparatuses discussed herein may be employed, for example, in the oil and gas industries. As one example, in a well that produces oil and water, or gas and water, the individual flows of each of the substances can be measured. Additionally, the individual flows of oil, water, and gas may be measured. In another example, in Steam Assisted Gravity Drainage (SAGD) systems, embodiments of the present disclosure may be used for determining, for example, steam to oil ratio.

Exemplary applications may include processing crude and gas flows, monitoring and detecting fluid or gas loss, monitoring the flow of cooling liquids, measuring discharge. In the wet gas industry, metering and measuring may be performed at the well head prior to the mixing of multiple gas streams, or thereafter. In diaries, the apparatus and methods of the present disclosure may be used, for example, to obtain estimates of fat, sugar, and protein content of milk.

Another environment in which this is applicable to is underwater oil exploration and production. For example, in addition to monitoring the flow of crude and gas, the flow of foam or corrosion inhibitors or other chemicals injected into the stream may be metered and/or measured.

Additionally, another environment to which this is applicable is oil extraction such as hydraulic fracturing, commonly known as fracking. In addition to metering and measuring activities, identifying the composition of substances can also be performed. In this regard, chemicals used in hydraulic fracturing may be damaging to pipelines and storage vessels, and further, could lead to pollution during burning.

To cope with such hazards, the method and apparatus described herein may be adapted to detect chemicals used during the hydraulic fracturing, including acids, salts, polyacrylamide, ethylene glycol, potassium carbonate, sodium carbonate, isopropanol, glutaraldehyde, lubricants, methanol, radionuclides, radioactive tracers, radioactive contaminants, etc.

It is noted that the term Doppler signal refers to any variation over time of the measured system response function.

As used herein, the singular form “a”, “an” and “the” include plural references unless the context clearly dictates otherwise. The use of the terms “at least one”, “one or more”, or the like in some places is not to be construed as an indication to the reference to singular only in other places where singular form is used.

As used herein the term “about” refers to ±10%.

The terms “comprises”, “comprising”, “includes”, “including”, “having” and their conjugates mean “including but not limited to”; and encompass the terms “consisting of” and “consisting essentially of”.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications, enhancements, and variations that fall within the spirit and broad scope of the appended claims. Thus, to the maximum extent allowed by law, the scope of the present disclosure is to be determined by the broadest permissible interpretation of the following claims and their equivalents, and shall not be restricted or limited by the foregoing detailed description.

The method and apparatus of the present disclosure, and aspects thereof, are capable of being distributed with a computer readable medium having instructions thereon. The term computer readable medium includes a single medium or multiple media, such as a centralized or distributed database, and/or associated caches and servers that store one or more sets of instructions. The term computer readable medium shall also include any medium that is capable of storing, encoding, or carrying a set of instructions for execution by a processor or that cause a computer system to perform any one or more of the methods or operations disclosed herein.

In a non-limiting exemplary embodiment, the computer readable medium can include a solid-state memory such as a memory card or other package that houses one or more non-volatile read-only memories. Further, the computer readable medium can be a random access memory or other volatile re-writable memory. Additionally, the computer readable medium can include a magneto-optical or optical medium, such as a disk or tapes or other storage device to capture carrier wave signals such as a signal communicated over a transmission medium. Accordingly, the disclosure is considered to include any computer readable medium or other equivalents and successor media, in which data or instructions may be stored.

It should be understood that the Abstract of the Disclosure should not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

1-75. (canceled)

76. A method of determining a value of a property of a material that flows in a conduit inside a microwave cavity, the method comprising:
exciting a plurality of excitation setups in the microwave cavity;
detecting parameters indicative of an electrical response of the microwave cavity to the exciting of the plurality of excitation setups in the microwave cavity; and
determining the value of the property based on the detected parameters by application of a kernel method.

77. The method of claim 76, comprising determining a time derivative of at least one parameter indicative of the electrical response of the microwave cavity to the exciting of the plurality of excitation setups in the microwave cavity, and determining the value of the property based on the time derivative determined.

78. The method of claim 76, comprising determining a time derivative of a scattering parameter Sᵢⱼ, and determining the value of the property based on the time derivative determined.

79. The method of claim 78, wherein i=j.

80. The method of claim 76, comprising reducing a dimension of an input vector using supervised or unsupervised learning, wherein the input vector includes values of parameters indicative of the electrical response of the microwave cavity to the exciting of the plurality of excitation setups in the microwave cavity.

81. The method of claim 76, comprising reducing a dimension of an input vector using supervised or unsupervised learning, wherein the input vector includes time derivatives of values of parameters indicative of the electrical response of the microwave cavity to the exciting of the plurality of excitation setups in the microwave cavity.

82. The method of claim 76, comprising reducing a dimension of an input vector using supervised or unsupervised learning, wherein the input vector includes combined parameters indicative of the electrical response of the microwave cavity to the exciting of the plurality of excitation setups in the microwave cavity.

83. The method of claim 76, wherein the exciting of the plurality of excitation setups comprises exciting through a plurality of feeds.

84. The method of claim 76, wherein excitation setups of the plurality of excitation setups differ from one another in at least one of a frequency or a feed.

85. The method of claim 76, wherein the parameters indicative of the electrical response of the microwave cavity to the exciting of the plurality of excitation setups in the microwave cavity include a scattering parameter S₁₁.

86. The method of claim 76, comprising combining parameters indicative of electrical response of the microwave cavity to the exciting of the plurality of excitation setups in the microwave cavity to obtain combined parameters, and determining the value of the property based on the combined parameters.

87. The method of claim 76, wherein the material is crude oil.

88. The method of claim 87, wherein the property is a volume fraction of water in the material.

89. The method of claim 87, wherein the property is a volume fraction of oil in the material.

90. The method of claim 87, wherein the property is a volume fraction of gas in the material.

91. The method of claim 76, wherein the property is a flow rate of the material.

92. An apparatus for determining a value of a property of a material that flows in a conduit inside a microwave cavity, the apparatus comprising:
a multi-mode microwave cavity having therein the conduit;
at least one feed, configured to feed the microwave cavity with RF radiation to excite multiple excitation setups in the microwave cavity;
a detector, configured to detect parameters indicative of an electrical response of the microwave cavity to radio frequency (RF) radiation fed to the microwave cavity; and
a processor, configured to determine the value of the property based on the parameters detected by the detector by application of a kernel method.

93. The apparatus of claim 92, wherein the at least one feed comprises a plurality of feeds.

94. The apparatus of claim 92, comprising a source of RF energy, configured to supply RF signals to the multi-mode microwave cavity through the at least one feed.

95. The apparatus of claim 94, wherein the processor is configured to control the source of RF energy to excite in the multi-mode microwave cavity a plurality of excitation setups that differ from one another in at least one of frequency and feed.

96. The apparatus of claim 93, wherein the apparatus is configured to excite in the multi-mode microwave cavity excitation setups through at least two of the plurality of feeds.

97. The apparatus of claim 93, wherein the processor is configured to receive input from the detector, determine a time derivative of the input, and determine the value of the property based on the time derivative so determined.

98. The apparatus of claim 97, wherein the input from the detector comprises values of a scattering parameter Sᵢⱼ, and the processor is programmed to determine the value of the property based on the time derivative determined.

99. The apparatus of claim 98, wherein i=j.

100. The apparatus of claim 93, wherein the processor is programmed to:
generate an input vector based on input received from the detector;
reduce a dimension of the input vector using supervised or unsupervised learning to obtained a reduced input vector; and
determine the value of the property based on the reduced input vector by application of a kernel method.

101. The apparatus of claim 93, wherein the processor is programmed to:
generate an input vector based on a time derivative of input received from the detector;
reduce a dimension of the input vector using supervised or unsupervised learning to obtained a reduced input vector; and
determine the value of the property based on the reduced input vector by application of a kernel method.

102. The apparatus of claim 93, wherein the processor is programmed to:
generate an input vector based on combined parameters, comprising combinations of parameters received from the detector;
reduce a dimension of the input vector using supervised or unsupervised learning to obtained a reduced input vector; and
determine the value of the property based on the reduced input vector by application of a kernel method.

103. The apparatus of claim 102, wherein the processor is programmed to generate the input vector based on a time derivative of the combined parameters.

104. The apparatus of claim 93, further comprising a dielectric attenuating conduit configured to attenuate electrical field exiting from the multi-mode microwave cavity.

105. The apparatus of claim 104, wherein the dielectric attenuating conduit is formed of glass fabric reinforced composite material.
